(19) United States
(12) Patent Application Publication
(10) Pub. No.: US 2011/0202039 A1
(43) Pub. Date: Aug. 18, 2011

(54) Method for Removing Kidney Stones and Endoscope Suitable for Said Method

(76) Inventor: Hansgeorg Schaaf, Reichertshausen (DE)

(21) Appl. No.: 13/058,786

(22) PCT Filed: Aug. 6, 2009
(86) PCT No.: PCT/DE2009/001131

§ 371 Date: May 4, 2011

(30) Foreign Application Priority Data
Aug. 11, 2008 (DE) 102008037260.9

(51) Int. Cl. A61M 1/00

(52) U.S. Cl. 604/540

(57) ABSTRACT

The invention relates to a method for removing kidney stones and to an endoscope for performing said method. According to the invention, the kidney stones are suctioned and moved into an access region, from which they can be removed in the conventional manner. The endoscope according to the invention is designed such that it can rinse and suction at the same time.

The invention relates to a method for removing kidney stones and to an endoscope suitable for said method.

Such an endoscope is, for instance, distributed by the applicant under the trademark

“Polyscope” and is described in DE 10 2004 005 709. This controllable flexible endoscope comprises a multi-lumen catheter probe in which at least an optical channel for accommodating optic, a working channel, a rinsing channel, and a control channel for accommodating a control element are provided. Via this control element it is possible to bend a distal probe element with a predetermined length or to twist same about a probe longitudinal axis, so that this therapeutic endoscope is adapted to be taken to an optimum operating position in a relatively simple manner. Such endoscopes are, for instance, used in ureteral endoscopy. Thus, these endoscopes are, for instance, used when removing kidney stones from the renal calyx or in the middle and lower ureter. Conventionally, in so doing the endoscope is positioned, and one tries to extract the kidney stones by means of a basket. Due to the extremely winding structure of the renal calyx which is difficult to access, this extraction of the kidney stones is extremely complicated, so that the person performing the endoscopy has to have great experience in order to locate the kidney stone, catch it with the basket, and remove it. There is a considerable risk of injuring the ureter in this process.

Contrary to this, it is an object of the invention to provide a method and an endoscope with which the removal of kidney stones is simplified.

This object is solved by a method with the features of claim 1 and by an endoscope with the features of independent claim 5.

In accordance with the invention, the endoscope is first of all introduced into a region of the ureter or of the renal calyx that is positioned in the direction of introduction in front of the kidney stone(s).

Subsequently, this region is rinsed so as to swirl up the kidney stones. The swirled up kidney stones are then suctioned by means of a vacuum source and are, by means of this vacuum, moved into a region that is easy to access for a tool, for instance, a basket. After having been moved to the access region, the kidney stones will be removed.

The endoscope suitable for such a method therefore has to comprise both a rinsing lumen and a suction lumen which are both controlled simultaneously or successively. In addition to the suction lumen and the rinsing lumen, the endoscope according to the invention is also equipped with an optic lumen and a control lumen.

In a particularly preferred solution the suction lumen and the rinsing lumen are arranged coaxially to each other.

It is preferred to design the suction lumen and the rinsing lumen with a suction probe that is introduced through a working lumen.

This suction probe may, for instance, be interspersed by a jetting lance, wherein a remaining annular space serves as a suction lumen.

The suction probe is preferably designed with a Y piece, wherein a rinsing line is connected to one connecting branch and a suction line is connected to the other connecting branch.

This suction probe may then be inserted into a working lumen of the endoscope. After the moving of the kidney stones into the access region, the suction probe is removed and the tool for removal of the kidney stones is introduced through the working lumen.

The method is particularly advantageous to perform if the region in which the kidney stones are arranged is sealed via the endoscope. This sealing may, for instance, be performed by means of a balloon that bulges out in radial direction from the outer circumference of the catheter probe and is in sealing contact with the ureter wall.

Other advantageous further developments of the invention are the subject matters of further subclaims.

In the following, a preferred embodiment of the invention will be explained in more detail by means of schematic drawings. There show:

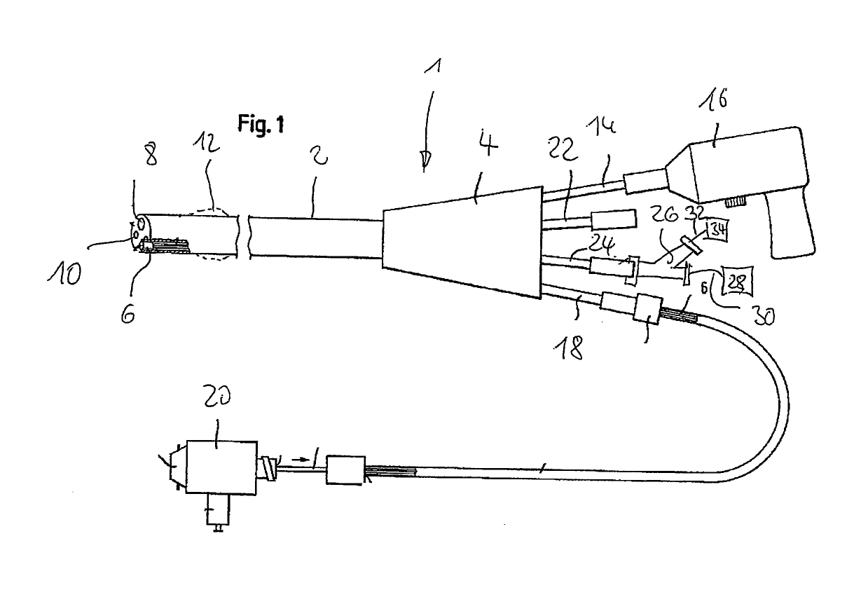

FIG. 1 a schematic diagram of an endoscope in accordance with the invention; and

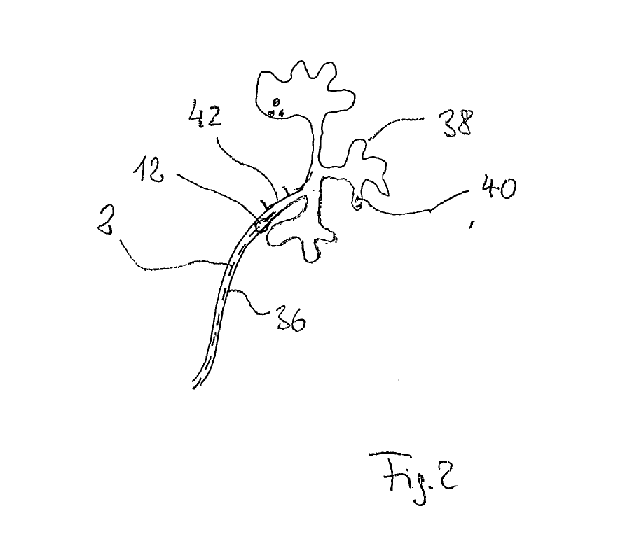

FIG. 2 a schematic diagram for explaining the method in accordance with the invention.

The flexible endoscope 1 illustrated in FIG. 1 comprises a multi-lumen catheter probe 2 at the proximal end portion of which there is attached a hub 4 at which the lumen exits are formed. In the illustrated embodiment, the catheter probe 2 is designed with an optic lumen 6, a working lumen 8, and a light lumen 10 as well as a control lumen. At the outer circumference of the catheter probe 2 there is further provided a balloon 12 that is adapted to be expanded in radial direction and that is indicated in dashes, said balloon 12 being, for instance, adapted to be expanded by the supply of air, wherein this supply of air may be performed through one of said lumens, for instance, through the optic lumen 6 closed toward the patient.

A handle 16 of a control means through which it is possible to deflect or twist the distal end of the flexible catheter probe 2 is fixed to the control lumen exit 14. The fiber bundles of the optic are guided out of the catheter probe 2 via an optic lumen exit 18 and are protected in this external region by a protective tube and connected to an optic base body 20 via which an ocular may be attached.

The optical fibers exit via the light lumen exit 22.

A tool or—as in the invention—a rinsing probe or jetting lance may be attached to a working lumen exit 24. In the illustrated embodiment, this rinsing probe is attached via a Y piece 26 with luer lock couplings, wherein a rinsing line 30 leading to a rinsing reservoir 28 is connected to the axial connection of the Y piece 26, and a suction line 32 that is connected to a suction or vacuum pump is connected to the inclined connection. This Y piece 26 is attached to the rinsing probe introduced into the working lumen 8, which has, however, a smaller diameter than the working lumen. The suction line 26 is then connected to the annular space between the inner circumferential wall of the working lumen 8 and the outer circumferential wall of the rinsing probe. Accordingly, it is possible to rinse and suction simultaneously or directly successively without a movement of the endoscope.

In accordance with the schematic illustration in FIG. 2, the catheter probe 2 is introduced by means of the control means through the ureter 36 into the renal calyx 38, and subsequently the renal calyx 38 and the portion of the ureter 36 opening therein are sealed against the bladder by the expanding of the balloon 12. The kidney stones 40 that are often to be found at places of the renal calyx which are very difficult to access are then swirled up by the rinsing probe with rinsing fluid and are suctioned into an access region 42 that is easy to access by means of the vacuum pump 34 that is actuated simultaneously or triggered after the rinsing process. After the kidney stones have accumulated in this access region 42, the rinsing probe is extracted from the working lumen 8, and a basket or some other tool is guided through the working lumen 8 into the access region 42 to receive the kidney stones and to extract them through the catheter probe 2.

The method according to the invention is extremely gentle for the patient since the endoscope movement is minimal. Furthermore, the work of the physician is facilitated quite substantially since the endoscope 1 merely has to be guided into the access region 42 and subsequently, during the performance of the suction process, there has to be monitored whether the kidney stones 40 accumulate in the desired region. This monitoring may be performed via the optic of the endoscope 1.

The invention relates to a method for removing kidney stones and to an endoscope for performing said method. According to the invention, the kidney stones are suctioned and moved into an access region, from which they can be removed in the conventional manner. The endoscope according to the invention is designed such that it can rinse and suction at the same time.

### LIST OF REFERENCE SIGNS

1 endoscope
2 catheter probe
4 hub
6 optic lumen
8 working lumen
10 light lumen
12 balloon
14 control lumen exit
16 handle
18 optic lumen exit
20 connecting piece
22 light lumen exit
24 working lumen exit
26 Y piece
28 rinsing reservoir
30 rinsing line
32 suction line
34 vacuum pump
36 ureter
38 renal calyx
40 kidney stone
42 access region

1. A method for removing kidney stones from a renal calyx or a ureter, comprising the steps of:
introducing a catheter probe in front of a region in which at least one kidney stone is positioned;
rinsing said region with a rinsing medium;
connecting a vacuum source to said catheter probe and moving said kidney stones from said region into a well accessible access region; and
removing said kidney stones from said access region.

2. The method according to claim 1, wherein the suctioning and rinsing is performed through a joint lumen of said catheter probe.

3. The method according to claim 1, wherein the removal is performed by means of a tool, preferably a gripper or a basket.

4. The method according to claim 1, wherein the region is sealed prior to rinsing or suctioning.

5. An endoscope, in particular for performing the method according to claim 1, comprising a rinsing lumen, an optic lumen, a control lumen, and a suction lumen.

6. The endoscope according to claim 5, wherein said suction lumen and said rinsing lumen are arranged coaxially to each other.

7. The endoscope according to claim 6, wherein said suction lumen and said rinsing lumen are designed in a rinsing probe that is introduced through a working lumen.

8. The endoscope according to claim 7, wherein a remaining annular space between said rinsing probe and said working lumen serves as a suction lumen.

9. The endoscope according to claim 8, wherein said rinsing probe is connected via a Y piece comprising a connecting branch for a rinsing line and a connecting branch for a suction line.

10. The endoscope according to claim 5, wherein an expandable balloon is provided for sealing at a catheter probe.

11. The endoscope according to claim 5, comprising a tool for removing said kidney stones through said working lumen.
